(19) United States
(12) Patent Application Publication
(10) Pub. No.: US 2018/0231555 A1
(43) Pub. Date: Aug. 16, 2018

(54) SYSTEMS AND METHODS FOR ISOLATING TARGET PARTICLES AND THEIR USE IN DIAGNOSTIC, PROGNOSTIC, AND THERAPEUTIC METHODS

(71) Applicant: APOCELL, INC., Houston, TX (US)

(72) Inventor: Darren W. Davis, Houston, TX (US)

(21) Appl. No.: 15/752,196

(22) PCT Filed: Aug. 9, 2016
(86) PCT No.: PCT/US16/46197

§ 371 Date: Feb. 12, 2018

Related U.S. Application Data
(60) Provisional application No. 62/203,900, filed on Aug. 11, 2015.

(51) Int. Cl. G01N 33/574; C12Q 1/6886; B03C 5/00

(52) U.S. Cl. CPC G01N 33/574; B03C 2201/26; B03C 5/005; C12Q 1/6886

(57) ABSTRACT

The present disclosure relates to an apparatus and methods for enriching and analyzing viable target particles of interest from a heterogeneous suspension containing target particles and non-target particles where the particles may be biological or non-biological. The method allows for the resulting target particles or a subset of target particles to undergo further analyses since the particles are not modified or labeled, which would typically interfere with further characterization. Another embodiment may be directed to a method of analyzing a fluid specimen from a subject suffering from a disease or condition comprising the steps of: obtaining a fluid specimen from a subject, culture, or animal model, where the fluid specimen comprises target cells of interest and non-target cells; adding said fluid specimen into a flow chamber; enriching viable target cells from non-target cells of the fluid specimen; and analyzing the viable target cells.

### BACKGROUND

More than 11 million people are diagnosed with cancer each year; it is estimated that there will be 16 million new cases every year by 2020 (Cho, W S C., Mol Cancer 2007; 6:25). Traditionally, pathologists have played a major role in the initial diagnosis of cancer, and in the morphologic classification and evaluation of the responsiveness of the patient to therapy, based upon analysis of tissue samples (i.e., serial biopsies).

Understanding molecular determinants of cancer disease and therapy response has grown in the past two decades to enable an individualized approach to patient treatment. However, current methods of examining the disease of individual patients are primarily based on tissue samples (computed tomography (CT)-guided biopsies or surgical material). These methods have several limitations that include invasive, risky collection procedures for the patient, limited accessibility of the tumor tissue, dependent upon the cancer type and location, and the use of a patient's archival tumor samples that may have been collected several years prior to metastatic relapse making the molecular information about the patient's tumor outdated. The combination of these factors results in less effective therapy for the patient and an unbeneficial economic impact to society. Furthermore, because of the nature of metastatic cancer, oncologists often have to wait several months before they can determine if a specific treatment is beneficial to the patient. Therefore, better tools are needed in oncology to support early detection of cancer, real-time monitoring, and provide physicians with the ability to tailor patient-specific therapy to improve the survival of cancer patients.

The vascular dissemination of tumor cells is a characteristic rate-limiting step of metastatic cancer progression (Fidler, I. J. (2003). Nat Rev Cancer 3(6), 453-458). Circulating Tumor Cells (CTCs) are cancer cells that have disseminated from a primary cancer site or its metastases found circulating in peripheral blood. Although the majority of cancer deaths are due to metastases, CTCs may actually be the germinating point for subsequent metastases. Accordingly, CTCs carry disease-relevant molecular information that can be present in the blood stream at a low frequency, as low as 1 cell in about 10 million peripheral blood mononuclear cells. The rare presence of CTCs introduces a significant challenge for their detection and analyses and requires a highly sensitive and highly specific means. Since CTCs make up a very small population among red blood cells and leukocytes and are normally absent from the peripheral blood of healthy subjects, they are increasingly used as biomarkers for metastatic cancers. (Pantel, K., Riethdorf, S. (2009). Nat Rev Clin Oncol 6(4), 190-191; Allard, W. J., et al. (2004). Clin Cancer Res 10(20), 6897-6904. CTCs offer a minimally invasive “liquid biopsy” to assess a patient's tumor in real-time, thereby enabling the profile of a patient's tumor so that oncologists can match the appropriate drug with the patient to improve treatment and overall response outcomes. CTC counts that may be identified by the conventional CK+/CD45-phenotype correlate negatively with progression free survival and overall survival in patients with metastatic colorectal, breast, prostate, or lung cancers (Budd, G. T. et al. (2006). Clin Cancer Res 12(21), 6403-6409; De Bono, J. S., et al. (2008). Clin Cancer Res 14(19), 6302-6309; Cohen, S. J., et al. (2008). J Clin Oncol 26(19), 3213-3221; Cristofanilli, M. et al. (2004). N Engl J Med 351(8), 781-791; Maheswaran, S., et al. (2008). N. Engl. J. Med 359, 366-377).

Based on developing evidence, CTC isolation from a blood sample may allow reliable early detection of cancer and its molecular characterization. This may thus provide a minimally-invasive method for monitoring the results of cancer therapy and guiding treatment. For example, a decreased Progression free survival (PFS) has been correlated to EGFR mutations found in circulating lung cancer cells Maheswaran, S., et al. (2008). N. Engl. J. Med 359, 366-377. By monitoring the reaction of circulating breast cancer cells to adjuvant chemotherapy enabled detection of patients at risk of early relapse Pachmann, K., J Clin Oncol 26(8), 1208-1215. There still remains, however, a need for CTC isolation technologies which can successfully isolate viable subsets of CTCs from a variety of cancers.

Despite the urgent need for alternative approaches, there is only one Food and Drug Administration (FDA) cleared test (CellSearch® by Veridex, LLC; Johnson & Johnson) on the commercial market that utilizes CTCs, but only as a predictor of survival for prostate, breast, and colorectal cancers. The major limitation with the CellSearch® test is that as an antibody dependent method, it has been cleared by the FDA for only three cancer types, and importantly, does not provide the ability to generate any molecular information about the cancer. This molecular CTC information is needed by the physician to provide the most effective therapy to the patient. In addition, other commercial CTC analytic technologies in development are limited in their ability to efficiently capture a sufficient number of viable CTCs from whole blood and from various types of cancer to be diagnostically and clinically useful. An adequate number of various subsets, ie, populations of CTCs is needed in order to perform the molecular based assays to profile a patient's tumor or cancer. Furthermore, many technologies rely on the fixation of cells for capturing limiting the ability to expand or produce cultures of cells in vivo or ex vivo models. Thus, the challenge of providing physicians and drug development companies conducting clinical trials in oncology with a broadly applicable and economical method, which allows rapid analyses of predictive markers on CTCs for diagnosis, prognosis, and therapy is not solved by currently available technologies.

More recently, there has been a significant advancement of understanding the molecular origins of different types of cancer and characteristics of tumor aggressiveness, based upon a major expansion of genomic and proteomic data. Cancer cells display a broad spectrum of genetic alterations that include gene rearrangements, point mutations, and gene amplifications, which lead to disturbances in molecular pathways regulating cell growth, survival and metastasis. When such changes manifest themselves in patients (from a small percentage to a majority of patients) having a cancerous tumor, or receiving treatment with a chemotherapeutic agent having a particular mechanism of action, discovery and quantification of these changes can be used to identify biomarkers for detecting and developing targeted therapies, and for predicting the clinical response to chemotherapeutic drugs used to treat the disease. The identification of new predictive biomarkers can provide invaluable assistance to clinicians in minimally-invasively and rapidly predicting a patient's response to therapy, selecting the best treatment modality, monitoring response to treatment over the course of therapy, as well as post-therapy, to thereby improve the likelihood of overall and recurrence-free survival. The advantages of the above cannot be understated.

Recent technologies have allowed the detection and isolation of circulating tumor cells (CTCs). CTCs are rare cells present in the blood in numbers as low as one CTC per 10⁶-10⁷ leukocytes. Historically, the detection and capture of such cells has been challenging (Gupta, et al., Biomicrofluidics 6, 024133 (2012)). Techniques currently used for CTC capture include immunomagnetic separation (Cohen, S. J., et al., J. Clin. Oncol. 26, 3213 (2008); Maheswaran, S., et al., N. Engl. J. Med. 359, 366 (2008), membrane filters (Desitter, I., et al., Anticancer Res. 31, 427 (2011), micro-electro-mechanical system chips (Nagrath, S., et al., Nature 450, 1235 (2007)), and dielectrophoretic field-flow fractionation (DEP-FFF) technology (Gupta, V., et al, ibid.).

Generally, CTC detection and analysis methods are composed of the following steps: an enrichment (isolation) process and a detection (identification) process (cytometric and nucleic acid techniques), which may or may not be separated from the enrichment. Genetic and molecular characterization of CTCs is typically conducted by fluorescent in situ hybridization (FISH), comparative genomic hybridization (CGH), PCR-based techniques, and biomarker immunofluorescent/immunohistochemical staining. Normally absent from the peripheral blood of a healthy donor, CTC counts have been described to correlate negatively with progression-free survival and overall survival in patients with metastatic, colorectal, breast, and prostate cancer (Gupta, V., et al., ibid.).

Common solid tumors from, such as, breast, lung, and colorectal cancers have been difficult to treat. Perhaps the difficulty is in part because they are heterogeneous, with each subset of patients having different molecular abnormalities, and sometimes within the tumor itself. Identifying relevant molecular CTC subtypes within heterogeneous diseases, and matching patients with appropriate targeted agents or combinations of them is crucial to future therapeutic progress.

Molecular profiling has special importance for cancer therapy. Mutation analysis is commonly performed on archived tumor tissue or on tissue from fresh biopsies, which can be a limiting factor for its use. A sufficient amount of tissue for analysis is not available for about 25% patients and the tissue is often archival in nature obtained several months to years at time of diagnosis. Therefore, development of new techniques that would allow oncologists to obtain the most amount of information from the least amount of biological material in real-time for monitoring during treatment is of paramount importance in biological analyses and more specifically, the further development of personalized cancer therapy.

In certain cancers, such as breast cancer, monitoring a patient's response to treatment is an essential component of therapy, since the degree of response can provide important prognostic information related to disease-free and overall survival. Histopathology provides an accurate assessment of treatment efficacy on the basis of the extent of residual tumor and regressive changes within the tumor tissue. However, only 20% of breast cancer patients achieve a pathologic complete response, a fact that necessitates methods for monitoring therapeutic effectiveness early during therapy (Avril, N. et al., The Journal of Nuclear Medicine, 50 (5) Suppl., May 2009, 55S-63S). Early identification of ineffective therapy may also be useful in patients with metastatic breast cancer and other types of cancer due to the number of palliative treatment options.

The number of CTCs from a patient has previously been correlated with patient survival. However, CTC isolation from a patient blood sample and subsequent molecular analysis of such cells have not been previously reported for the prediction of responsiveness of a patient to treatment with a particular type of chemotherapeutic agent. Nor have such analyses been widely used to provide a minimally invasive method to predict, guide, and monitor the results or progress of cancer therapy.

New methods for predicting therapeutic effectiveness prior to and over the course of therapeutic treatments of various cancers, especially methods that are rapid, minimally invasive, and available at an early stage of treatment, can help to individualize and guide treatment, avoid ineffective chemotherapies or drug treatment, provide near real-time analyses, and allow early detection in patients at risk for early relapse. Thus, there remains a need to provide, among other things, new and improved methods for the isolation of target particles that allow for further molecular analyses, where the methods are rapid and the resulting isolated target particles are viable and not altered in such a way that would disrupt or prevent further analyses. In particular, methods of isolating biological particles of interest, such as CTCs, which allow for early assessment and prediction of the efficacy of cancer treatment regimens, particularly in patients undergoing therapy, including, for example, traditional chemotherapy, molecularly targeted agents, and immunotherapy, where the sample CTCs are easily obtainable or non-invasively obtained, are desirable.

A new paradigm for therapeutic decision-making strategies has been developing over the last two decades in view of targeted therapeutics and molecular profiling of cancer biopsy tissues. Analyses of CTCs allows for the therapeutic management of cancer based on molecular characteristics of cancer in its existing state in real time. In contrast, tissue biopsies do not occur in real time, rather a tissue biopsy is taken at a specified time and then later analyzed.

Molecular analyses are routinely performed on resected tumors, small biopsies or cytological samples (e.g., fine needle aspirates) from the primary site for mutation detection which provide guidance for classifying or stratifying patients for targeted treatments. However, both the quality and quantity of tumor material used for analyses often hinder detection of these genetic abnormalities or mutations. As briefly mentioned, tumor biopsy material provides a biological limitation on the genetic alterations present at the time of collection, i.e., the biopsy provides a “snapshot” frozen in time. One of the disadvantages of archival biopsy tissue is the inability to provide information on mutations that develop during the metastatic process or materialize in response to pharmacological treatments. Surgery is rarely a component of treatment in advanced or metastatic patients, specifically in the case of NSCLC (Non-Small Cell Lung Cancer) patients. Even if tumor material is available for retrieval, obtaining a sample using thoracentesis, bronchoscopy, or similar procedures often presents a risk of co-morbidity in view of the nature of the tissue and issues related to accessibility.

Significant advances have also been achieved in antenatal screening for fetal aneuploidy. Fetal aneuploidy is defined as an abnormal number of chromosomes instead of the usual diploid complement of 46 chromosomes. For example, one single additional chromosome (termed trisomy) is an important cause of congenital malformations as well as of congenital mental retardation, such as Down syndrome. Edwards syndrome and Patau syndrome are the most common trisomies, and are lethal and affect approximately 1 in 3000 and 1 in 5000 live births, respectively. The majority of infants with Edwards syndrome die by one year of age, while the majority of infants with Patau syndrome die by 3 months of age. Another form of trisomy is present in approximately 1 in 800 to 1 in 900 live births, and is associated with congenital heart defects, most commonly the atrio-ventricular canal defect.

Determination of aneuploidies is thus essential during the prenatal period, especially for women of advanced maternal age (i.e., at least 35 years old at the expected date of delivery) and those having had a previous child or pregnancy with chromosomal abnormalities or multiple congenital malformation, or with abnormal ultrasound findings and a positive screening test result for abnormal serum biochemical markers.

Although the invasive method of amniocentesis is still employed, non-invasive prenatal diagnosis is now routinely performed on maternal blood by standard cytogenetic techniques for numerical and major structural chromosomal abnormalities. Indeed, cytogenetic analysis is currently held to be the gold standard for prenatal diagnosis. However, it has the major disadvantage that up to 14 days or more of cell culture is required before analyses can be made on a sufficient number of fetal cells. Cytogenetic techniques can also fail because of cell culture ‘crashes’, and contamination with maternal cells can cause false negative and false-positive results. Therefore, there is a need for prenatal genetic screening that is highly sensitive, accurate, and non-invasive or substantially non-invasive.

Therefore a need exists for an alternative, less invasive or substantially non-invasive, highly efficient, and highly pure recovery means resulting in high viability of targets of interest from a specimen to screen patients who may benefit from potentially effective therapies as well as monitor their response to treatment and to understand mechanisms of resistance. Additionally, there is a need for a method of isolating and capturing CTCs by a non-invasive means to enable measuring tumor burden (enumeration) and sampling for molecular markers useful in the prognosis, monitoring treatment responsiveness, aiding in treatment decision-making, and potentially, even early detection of a particular sub-population of CTCs in a sample.

### FIELD OF THE INVENTION

This invention generally relates to the field of microfluidic dielectrophoresis and more specifically to methods of isolating, separating, and capturing a population of particles for prognostic, diagnostic, monitoring, and genetic disease screening methods based on the identification and subsequent processing and/or analyses of target particles. In particular, embodiments of the invention generally relate to diagnostic, monitoring, and screening methods that find application areas related to, but not limited to, stem cells, fetal cells, cells for regenerative medicine, cell therapy, infectious disease, and oncology.

### BRIEF SUMMARY OF THE DISCLOSURE

Aspects of the invention are generally directed to methods of isolating target cells by minimally invasive means, obtaining viable target cells, and the subsequent analyses of the isolated cells. In one aspect, embodiments of the invention are directed to methods for the screening, diagnosis, prognosis, treating, and monitoring of a specimen containing a sample of cells of interest or target particles, where the isolated cells are unaltered and viable.

Another aspect of the invention is directed to methods for treating diseases using isolated cells or particles that may be altered, unaltered or subsequently fractionated after isolation of specific subsets for the generation and use of biological or genetic material including various types of proteins, RNA and DNA. This biological or genetic material may be modified and/or returned to the patient by, for example, transfecting into cells.

One aspect of the disclosure may be directed to a method for the screening, diagnosis, prognosis, or monitoring of a subject for diseases, conditions, or illnesses, particularly genetic diseases, comprising the steps of: obtaining a fluid specimen from a subject suffering from or suspected of having a disease or condition, where the fluid specimen comprises a suspension of target particles of interest and non-target particles; determining or utilizing pre-determined dielectrophoretic (DEP) crossover frequencies of the target particles of interest; infusing the fluid specimen into a dielectrophoretic flow chamber; diffusing the fluid specimen; separating the target particles from the non-target particles under continuous flow dielectrophoresis field-flow (FF) assist principals; and isolating or obtaining the target particles of interest. The target particles of interest are viable and undergo further analyses to characterize the target particles. The analyses of a subject specimen consequently provide a prognosis, diagnosis, or means for monitoring a subject's condition, where the subject specimen is an organic fluid taken from a subject and contains cells of interest. For example, the fluid specimen may be obtained from the peripheral circulation of subject or bone marrow or other bodily fluid or from cultures outside the body. The isolated target particles may be, for example, cells, such as CTCs or a subset of CTCs, in a viable state. The CTCs may also be in an altered state if the subject has undergone treatment affecting the cells, such as chemotherapy or the like.

Another aspect of the disclosure is directed to isolated target particles of interest, including but not limited to for example, cancer cells, different stages of cancer cells, circulating tumor cells, disseminating tumor cells, fetal cells, stem cells, cells for regenerative medicine, cell therapy, infectious disease, supporting cells for tumor assault that are abnormal, and the like. Obtaining a highly pure or enriched sample with target particles or cells of interest that are viable is significantly beneficial and required for subsequent analyses and culturing. The isolated target particles may be a homogenous population of pure or enriched target particles. These populations of target particles or cells are preferably substantially pure for specific molecular applications. After the enrichment step of a target particle population, about 1% to about 100%, a purity of about 80%, about 85%, preferably about 90%, more preferably about 95%, and most preferably about 100% is highly desirable. Moreover, the enrichment step is extremely sensitive, such that in a fluid specimen, the high sensitivity allows for at least one target particle of interest to be obtained, collected, enriched, or isolated from the non-target particles. Furthermore, specific frequencies may be applied to the fluid specimen which enable the enrichment of specific, desired target cells or populations of target cells, which may be subsequently used for further analysis, including but not limited to diagnosis, prognosis, effective treatment, screening, and the like.

Further aspects of the invention include methods utilizing a microfluidic system for dielectrophoretic isolation of target particles or cells, preferably through the use of a microfluidic or flow chamber. Target particles, such as cancer cells, and normal, healthy, non-cancerous cells are known to have differences in their dielectrophorectic properties. Thus, isolation of the target particles of interest occurs based on the differences in parameters of target particles of interest in a fluidic state versus non-target particles. Non-limiting examples of the parameters used for DEP, DEP field-flow assist, or hybrid DEP methods such as DEP-FFF, DEP-Acoustophoresis, DEP-Magnetophoresis, and DEP-Electrowetting on Dielectric (EWOD) for the isolation of the particles of a fluid specimen include membrane surface charge, membrane capacitance, frequency, mass density, morphology, electrical properties, chemical properties, mechanical properties, expression of surface antigens, expression of intracytosolic antigens, dielectric properties, magnetic properties, optical properties, geometrical properties, or combinations thereof.

Yet another aspect of the inventive methods is the label-independent means for capturing target particles of interest. For example, CTCs for a variety of cancer types and stages of cancer may be captured without the requirement of labels, probes, markers, antibodies, or the like. However, diagnostic cells may be labelled. Particularly useful are antibodies for specific markers to identify certain subsets of cells required for diagnostic monitoring or subsequent treatment modalities. For example, at least one type of antibody may recognize antigens for non-limiting examples of epithelial, epithelial mesenchymal, mesenchymal, stem cells, and the like.

In a further aspect, the inventive method may include at least one enrichment step performed in combination with the isolation step within the same microfluidic device used for performing the isolation step. Enriching at least one population of cells of the fluid specimen, such as for example, at least one type of circulating tumor cells, disseminated tumor cells comprises at least a positive and/or negative selection using magnetic beads coupled to antibodies.

Another aspect may be directed to inventive methods that preserve cell viability, thereby enabling developing cell cultures for drug efficacy testing and development of patient-derived xenograft (PDx) models. Isolating or enriching cells or fractionated subsets of cells using DEP, DEP field-flow assist, or hybrid DEP techniques allows for further characterization of protein, DNA, or RNA content. Analyses may include, but are not limited to sequencing, microsatellite analysis, comparative genomic hybridization (CGH), array CGH, end-point PCR, real-time PCR, methylation analysis, such as, quantitative methylation analysis with pyrosequencing, bisulphite-genomic-sequencing PCR (BSP), and methylation-specific PCR (MSP), digital PCR, and Next-gen sequencing.

Yet a further aspect may be directed to isolating fetal cells, including fetal stem cells, from a maternal blood specimen for pre-natal testing, including but not limited to genetic and birth defects, chromosomal abnormalities, Down syndrome, Sickle cell disease, Cystic fibrosis, Muscular dystrophy, Tay-Sachs, neural tube defects, such as, spina bifida and anencephaly, and the like.

In another aspect, methods of the invention may utilize a microfluidic device having multiple different chambers that are separated from one another and yet fluidly connected, delimited on at least one face by an individual chip or by several separate chips. Having multiple chambers improves separation efficiency by separating cells in a first chamber using, for example, a frequency value 1, electrode spacing and width of certain values, and then further subfractionating in a second chamber using a different frequency and electrode geometry. The first chamber may employ straightforward DEP, or DEP-FFF, for example, and in the subsequent chamber a hybrid DEP method such as DEP-acoustophoresis or magnetophoresis could be employed.

### BRIEF DESCRIPTION OF THE DRAWINGS

FIG. 1A shows a flowchart detailing a preferred method embodiment of the invention.

FIG. 1B shows another flowchart detailing a preferred method embodiment of the invention exemplifying enriching and analysing cancer cells.

### DETAILED DESCRIPTION OF THE INVENTION

One embodiment of the present invention is to provide methods for isolating target particles and their subsequent analyses, where the target particles are viable obtained using minimally or substantially non-invasive means.

Briefly, embodiments of the disclosed methods of isolating target particles from a suspension containing target particles and non-target particles comprise steps of determining a dielectrophoretic (DEP) crossover frequency of the target particle of interest; infusing the suspension into a flow chamber; diffusing the suspension; separating the target particles from the non-target particles under DEP or DEP Field-flow assist; and isolating or obtaining the target particles. Further analyses of the obtained target particles may be performed to characterize the target particles. The analyses of a subject specimen consequently provide a prognosis, diagnosis, means for monitoring a subject's condition, or treatment, including but not limited to cell therapy, regenerative medicine, and the like, where the subject specimen is an organic fluid taken from a subject and contains cells of interest. The isolated target particles may be cells, such as CTCs or a subset of CTCs in an unaltered, viable state. Non-limiting examples of a subset of cancer cells may include those populations of cells that have a potential for clonogenecity, tumorgenicity, or migration invasion.

### Fluid Specimen

Another embodiment of the present invention is directed to a fluid specimen which may be a non-biological fluid or a biological fluid. The fluid specimen comprises a suspension of particles of interest or target particles, preferably target cells of interest, and non-target particles or cells. Non-limiting examples of biological or organic fluids may include a variety of body fluids, such as for example, blood, bone marrow, urine, ascites, mucus, exosomes, cerebrospinal fluid (CSF), and the like. Non-biological fluid specimens may include any liquid or fluid suspension comprising inorganic matter, minerals, crystals, colloidal particles, conductive particles, semi-conductive particles, insulating particles, gas bubbles, and the like.

Organic fluid specimens are preferably obtained from a subject suspected of having or known to have a particular disease, condition, or illness, such as, a genetic disease or disorder, or healthy, normal subjects who undergo preventative screening tests. The fluid specimen, if biological or organic, may be obtained, preferably in a non-invasive, substantially non-invasive, or less-invasive manner, from a subject suffering from or suspected of having a disease or condition, such as a genetic condition. The subject is preferably a mammal, most preferably human, and may also be an animal, including but not limited to cows, pigs, horses, chickens, cats, dogs, rats, mice, guinea pigs, etc.

When the cells are isolated, the cells are unaltered, unmodified, and unlabeled with ligands, antibodies, probes, stains, or the like. Accordingly, the cells or population of cells, are viable allowing for additional cell culturing and testing. Although the preferred methods are directed to isolating cells or biological matter without modification or alteration, the disclosed processes may be similarly used to isolate modified or altered biological cells.

The subject may have or suffer from, for example, a cancer, and by isolating the CTCs of the specific cancer from the other components found in a blood specimen, such as for example, plasma, red blood cells, peripheral blood mononuclear cells (PBMCs), white blood cells, and platelets, the highly pure and viable isolated CTCs may be further analyzed for determining the subject's response to cancer treatments, identifying the specific stage of the cancer, therapeutic and diagnostic purposes, and the like.

The organic fluid or corporeal fluid is obtained from a subject in the form of a specimen which has a high probability of containing the cells of interest. The organic fluid may be obtained from a subject by a non-invasive or substantially non-invasive manner, for example, directly by peripheral blood flow, bone marrow, urine, exosomes, or indirectly, such as for example, by trypsinizing a tissue from a lymph node. In one embodiment, the corporeal fluid may be peripheral blood, drawn in a substantially non-invasive way from the subject or patient suffering from a particular disease or disorder, in accordance with techniques commonly known and used.

The particles of interest may be biological cells, and the cells of interest are, for example, circulating tumor cells (CTCs). Another embodiment may be directed to an organic fluid that is blood obtained from bone marrow, and the target cells or cells of interest are disseminated tumor cells (DTC). Other non-limiting examples of biological particles in a biological fluid specimen include: cell organelles, cell aggregates or clumps, maternal/fetal cells, progenitor cells, stem cells, endothelial cells, epithelial cells, growth factor receptor expressing cells, cytokeratin expressing cells, cells expressing disease-specific proteins, bacterium, protozoans, viruses, and the like, or combinations. Also encompassed are cancer cells, such as but not limited to CTCs, cancer stem cells, disseminating tumor cells (DTC), non-epithelial cancers or cancers with low or negative epithelial cell adhesion molecule (EpCAM) expression, or metastatic cells that become tumors for specific cancers including various types of breast cancer, colon cancer, ovarian cancer, prostate cancer, lung cancer, pancreatic cancer, liver cancer, medulloblastoma, melanoma, glioblastoma, and the like. The biological fluid specimen in embodiments of the disclosure comprises a suspension of at least one target particle of interest, or preferably a population of target particles. The suspension may contain a mixture of cell types, such as for example, fetal nucleated red blood cells in a mixture of maternal blood, cancer cells such as breast cancer cells in a mixture with normal, healthy, non-cancerous cells, or red blood cells infested with malarial parasites.

Particles found in a specimen, such as an organic fluid, may include but are not limited to biological particles, e.g., tumor cells, fetal cells, stem cells, and non-biological particles. The specimen may be taken from a subject at any time, for example, before diagnosis of a genetic disease or during treatment for cancer. The particles or cells of interest may be detected using any one of several known appropriate techniques of analyses, which can provide indications of a diagnostic or therapeutic nature on a pathological condition of the subject from whom the specimen was obtained.

A cell as used here may be understood to include a single cell. Isolation of an aggregate of single cells forms a subset or subpopulation of cells. The cells of interest may not be individually isolated, but bound to one or more than one other cells, including tumor cells or non-tumor cells. For example, cells can be tumor microclusters or embryoid bodies (EBs) which are three-dimensional aggregates of pluripotent stem cells. The DEP cross-over frequency for a cell aggregate or a cell doublet, cell triplet, etc. differs from that of a single, isolated cell. This requires a different DEP protocol for separation than for a single cell. There is also a possibility that analysis may be of a part of a cell, such as for example, an isolated nuclei obtained after cell lysis in the flow chamber.

Embodiments of the present disclosure are also directed to a modified or improved dielectrophoretic method using an apparatus for discriminating between particulate matter of interest and solubilized matter of different types in a fluidic suspension. The apparatus and method of the disclosure integrate software, electronics, hardware, and fluidic systems to obtain a sub-population or sub-fraction of particles from a suspension of homogeneous particles. The technology disclosed here is a new and improved approach that permits the attainment of target particles of interest from other non-target particles and allows further analyses of the target particles of interest. The target particles and the non-target particles are preferably free from labels or modification, i.e., for example, antibody-free or magnetic bead-free, thereby allowing further unobstructed target particle analyses.

Dielectrophoresis (DEP) and hybrid DEP Field-Flow (FF) assist principles are used to sort cells with distinct biophysical characteristics by exploiting dissimilarities in the frequency-dependent dielectric properties of biological and non-biological particle types from characteristics including, but not limited to: size, morphology, and electrical characteristics. Briefly, the technology is based on DEP separation, or the difference between dielectrophoretic forces exerted on different particles in non-uniform electric fields. DEP migration utilizing DEP forces results in either the attraction or repulsion of different particle types when towards or away from electrodes, respectively. These opposing forces are known as positive and negative DEP, respectively. The technology combines the competing interaction between DEP forces, sedimentation forces and hydrodynamic lift forces and results in separation of target and non-target particles. Particles influenced by negative DEP forces are eluted by fluid flow; whereas, particles that are attracted by positive DEP forces are drawn toward the electrode surface and subsequently skimmed into a collection port.

The basis for the DEP field-flow assisted technology of the disclosure is the application of an alternating current (AC) voltage to induce DEP forces to differentially drive particle types of different dielectric properties into different heights within a microfluidic, laminar fluid flow chamber. In this DEP field-flow assisted configuration, the controlling DEP force acts perpendicular to the flow direction and works in conjunction with sedimentation and hydrodynamic forces to effect separation of the target particles or non-target particles from a mixed particle suspension. The DEP force (FDEP) acting on a spherical particle of radius r, suspended in a liquid medium of absolute permittivity c, is given by the relationship of equation (1).

F_{DEP}=2πεₛr³Re(f_{CM})∇E² (1)

where E is the root mean square (RMS) value of the applied electric field and ∇ is the gradient operator. Re(fCM) is the real part of the Clausius-Mossotti factor that defines the effective polarizability of the cell relative to that of the suspending medium.

Markx et al (Journal of Physics D: Applied Physics, Vol. 30, 1997, p. 2470) and Huang et al. (Biophysical Journal, Vol. 73, 1997, p. 1118) modified equation (1) to describe the vertical component of the DEP force averaged along a horizontal plane, acting on a particle at a height, h, above the electrode plane, which is given by equation (2).

F_{DEP}(h)=2πεₛr³p(f)Re(f_{CM})q(h)V² (2)

The factor p(f) defines the frequency-dependent electrode polarization which becomes an important factor at low frequencies. V is the applied RMS voltage and q(h) reflects the height dependence of the vertical DEP force component, which above a certain levitation height can be taken to decrease smoothly as a function of increasing levitation height. At low levitation heights, local values of the vertical component of the DEP force are determined by the factor ∇E² in equation (1), with maxima occurring above electrode surfaces.

If the target particles are biological cells in an embodiment, the flow chamber enables separation of these cells based on viability. This is a key difference compared to other non-DEP technologies, which isolate both dead and viable cells and other foreign bodies yielding a less pure sample. In embodiments of the improved method described here, pure subsets of cells, such as for example, cancer stem cells, can be isolated by the chamber and used for downstream diagnostic purposes. Whereas, other technologies cannot be applied to diagnostic or therapeutic applications because the resulting sample contains a mixture of cells including dead cells, and dead cells are not relevant for medical testing or clinical use.

The inventive technology only isolates target particles of interest or viable cells (depending on an intact cell membrane), thereby creating a pure or substantially pure sample of target particles or cells of interest. Embodiments of the invention may be directed to an isolated or enriched sample of target particles or cells of interest having a purity of about 1% to about 100%, preferably greater than about 85%, greater than about 90%, preferably greater than about 95%, and most preferably greater than about 100%, and/or sufficient sensitivity to isolate at least one target particle of interest. The inventive method is highly sensitive to separate or obtain at least one target particle, preferably a homogeneous population of target particles. The pure sample enables subsequent discovery and molecular characterization for therapeutic or diagnostic and prognostic use of the target particles or cells of interest.

### Target Particles and Non-Target Particles

The fluid specimen comprises target particles and non-target particles. The target particles of interest must be detectably unique from the non-target particles. Embodiments of the disclosure may be directed to particles of interest that may be isolated from the particles not of interest based on at least one parameter characterizing the target particles and/or non-target particles. Non-limiting examples of these particle characteristics include: mass density; morphology; size; shape; electrical properties; chemical properties; mechanical properties; dielectric properties; magnetic properties; optical properties; geometrical properties; expression of surface antigens; expression of intracytosolic antigens; and combinations thereof. With respect to the morphological properties, the size of the particles may range from about 10 nanometers to about 1 millimeter, preferably about 5 micrometers to about 20 micrometers which reflect the sizes of biological molecules such as, for example, proteins, DNA, and RNA. Further to biological molecules, additional non-limiting examples of particles include chemical molecules, subpopulations of molecules, cells, cell aggregates, viruses, plasmids, bacteria, protozoans, embryos, or other small organisms.

Other embodiments may be directed to methods for differentiating solubilized matter including but not limited to, a molecule or molecular aggregate, such as, for example, proteins or nucleic acids, including DNA or RNA or exosomes.

Since the target cells are not modified (i.e., no labelling or fixing) through the disclosed process, the resulting target cells can be further cultured and analyzed without obstruction for complete cell characterization. According to embodiments of the invention, various types of analyses can be performed on the recovered tumor cells that enable genetic or chromosomal characterization thereof, such as, for example, one or more of RNA, DNA, or protein analysis or other elucidating techniques.

### Methods of Obtaining or Isolating Target Particles

Embodiments of the disclosure are generally directed to methods of obtaining viable target particles of interest from a suspension of target particles and non-target particles in a fluid specimen. One embodiment comprises the isolation of biological particles, where the particles are cells and the sample suspension contains both target cancer cells and non-target, non-cancerous healthy blood cells. Isolating, enriching, or subfractionating means essentially separating one population from another population. In another embodiment, isolating may be used interchangeably with enriching, where enriching a population of target cells results essentially in a population of target cells with minimal contamination of other non-target cells, e.g. or where the population is viable versus non-viable or where the population carries specific genetic material applicable for the intended application (e.g., FISH, sequencing, etc). A further embodiment may be directed to the isolation or enrichment of rare cells, in particular circulating tumor cells, disseminated tumor cells, stem cells, fetal cells, epithelial cells, mesenchymal cells, epithelial-mesenchymal transition cells, or any other cells that may be useful in cell therapy, regenerative medicine, screening, diagnosing, or prognosing, or therapy, obtained preferably by a non-invasive or substantially non-invasive means from a subject.

The dielectrophoretic isolation of fetal cells advantageously avoids the use of invasive amniocentesis and also reduces the problems with current non-invasive cytogenic techniques by providing enriched fetal cell samples that do not require cell culture. The DEP-isolated fetal cells may preferably have low contamination of maternal cells. The differences between fetal cells and maternal cells allow for isolation. Differences between the cells include but are not limited to fetal red blood cells possessing a cell nucleus and having a slightly larger size and shape compared to maternal red blood cells. Moreover, the fetal cells possess different mechanical and other physico-chemical properties to maternal blood cells. These differences can be exploited by the DEP field-flow assist technology used here to provide pure samples of fetal cells available for immediate analysis of fetal aneuploidy.

An apparatus utilized for isolating, separating, and collecting cancerous cells may be modified from the apparatus or device including a flow chamber, which is conventionally used in DEP-FFF. One embodiment of the disclosure relates to an apparatus where the cancerous cells are attracted by positive DEP forces towards the electrode plane, and thus away from the bulk of the sample blood cells that are levitated by negative DEP into the fluid flow velocity profile. This is referred to as DEP Field-Flow assist separation, as compared to DEP-FFF where all of the cells are levitated at different heights into the fluid flow stream and appear at different positions along the effluent fluid flow stream. This inventive DEP-Field Flow assist method is accomplished by applying the voltage signal at a frequency in between or intermediate to the so-called DEP crossover frequencies of cancer cells and peripheral blood mononuclear cells (PBMCs).

The DEP crossover frequency is the point where the DEP force acting on a particle transitions from a negative to a positive polarity. The DEP crossover frequency may be determined using the technique disclosed in Provisional Patent Application No. 61/977,356, filed on Apr. 9, 2014 and entitled, “System and Method for Determining Dielectrophoresis Crossover Frequencies,” which is incorporated herein by reference. Properties exploited by DEP-fluid field assist include the difference between cancer cells and normal blood cells (Sangjo Shim et al. Biomicrofluidics, 7, 011808, 2013). After determining the crossover frequency of a target particle of interest, a frequency is applied in order to enrich a homogeneous population of target particles.

### Enrichment

The step of enriching a fluid specimen in a population of cells comprising target cells of at least one type may be performed by a method of consecutive steps by means of a selection of cells made on the basis of at least one parameter. Enrichment as used here is directed to the separation of target particles from non-target particles and then additively combining the target particles to become aggregated or enriched in a group or homogeneous population of target particles. In one embodiment, the fluid specimen may be enriched by undergoing DEP Field-flow assist to isolate or obtain a homogeneous population of target particles of interest.

Where an enrichment step is provided in embodiments of the invention, the target cells may be enriched according to any of the techniques known in the art. These techniques may preferably be automated. The target cells may be enriched on the basis of biological properties or physical properties. Non-limiting examples of physical properties may include size, density, electrical charge, optical properties, surface antigen expression, intracytosolic antigen expression, deformability, or the like, and combinations thereof. Examples of enriching techniques may include but are not limited to selective lysis of, for example, erythrocytes not of interest, filtration based upon the dimension of the cells, which may be obtained by photolithographic micromachining or other techniques, such as track-etched membranes, depletion or magnetic enrichment, immunomagnetic separation using for example immunomagnetic beads for positive selection which may be performed using beads labeled with specific antibodies for the population of cells to be obtained, or with negative selection by depleting the cell populations that are not of interest, combining positive and negative selection thereby increasing the specificity, and in which the two types of selection can be coupled in order to increase the specificity of the procedure, flourescence activated cell sorting (FACS) of fluorescently labelled cells labelled where the label is a specific fluorescent antibody; dielectrophoresis by for example dielectrophoretic-activated cell sorter (DACS), or centrifugation on a FICOLL® density gradient or PERCOLL®, solid-phase, immunoseparation using microfluidic systems presenting surfaces coated with specific antibodies for epithelial receptors (e.g., EpCAMs). One embodiment may be directed to a method of obtaining a fluid specimen, enriching the fluid specimen, applying DEP field-flow assist technology to obtain a population of viable target cells of interest for further analyses.

In another embodiment, more than one enrichment step may occur. Embodiments of the disclosure may be directed to an isolating step preceded by centrifugation on a density gradient or alternatively separation of whole blood. Moreover, target cells may be further enriched by positive or negative selection of the cells from a suspension of mononucleated cells. This additional enrichment step may comprise selecting cells based on the expression or lack of expression of a specific antigen as analyzed by a technique including but not limited to magnetic-activated cell sorter (MACS), DACS, FACS, EasySep™ (StemCell™ Technologies), or using paramagnetic beads that do not require the use of a specific column but may be used with wells or test tubes, such as for example anti-EpCAM Dynabeads.

Embodiments where a fluid specimen containing target cells of interest may be added or inserted in a microfluidic device including a flow chamber may enrich and isolate the target cells in an automated or semi-automated, non-manual way. For this purpose, it is possible to use dielectrophoretic isolation (using for example, the techniques described in WO/2015/031586 (PCT/US14/53107, “Method and apparatus for isolation, capture and molecular analysis of target particles”) and PCT/US2015/023971, “System And Method For Determining Dielectrophoresis Crossover Frequencies” as incorporated in their entirety here).

One embodiment of the invention envisages a process including one or more of the following whole blood pre-processing steps to remove, separate, or isolate non-target particles, such as red blood cells or cellular debris, from the target particles of interest. Non-limiting examples of pre-processing methods include a standard gradient based erythrocyte separation using FICOLL®, LEUCOSEP®, density gradient centrifugation, lysing red blood cells, centrifugation, and the like, or combinations thereof, which can isolate, separate, or remove cells not of interest.

Another preferred embodiment may be directed to a method of using a whole blood fluid specimen without pre-processing, such that minimal loss of target particles occurs. One pre-processing step may include lysing non-target particles or cells such as red blood cells. However, when lysing red blood cells, target cancer cells are inadvertently lost during the subsequent centrifugation step. Whereas, by bypassing the pre-processing step altogether, there is no loss of the desired target cells when enriching a whole blood specimen or sample. Furthermore, since many pre-processing methods occur prior to enrichment in a flow chamber, by performing the enrichment step without pre-processing in the flow chamber, more target cancer cells may be recovered. Another embodiment may include removing non-target cells by lysing the specimen in the flow chamber, which does not affect the enrichment of target cells. In an alternative, enrichment occurs initially or pre-enrichment to eliminate the erythrocytes by selective lysis of erythrocytes, which exploits chemical properties of the cells. Another method may utilize CD45 MACS depletion for bulk depletion of cells that are positive for CD45, which is a leukocyte common antigen and expressed in nucleated hematopoietic cells.

Another embodiment of the disclosure is directed to biological particles, where the particles are cells and the sample suspension contains both target cancer cells and non-target healthy blood cells. An apparatus utilized for isolating, separating, and collecting cancerous cells may be modified from the apparatus conventionally used in DEP-FFF. One embodiment of the disclosure relates to an apparatus where the cancerous cells are attracted by positive DEP forces towards the electrode plane, and thus away from the bulk of the sample blood cells that are levitated by negative DEP into the fluid flow velocity profile. This is accomplished by applying the voltage signal at a frequency in between or intermediate to the so-called DEP crossover frequencies of cancer cells and peripheral blood mononuclear cells (PBMCs).

The DEP crossover frequency is the point where the DEP force acting on a particle transitions from a negative to a positive polarity. The DEP cross-over frequency may be determined using the technique disclosed in Provisional Patent Application No. 61/977,356, filed on Apr. 9, 2014 and PCT/US2015/023971 both of which are entitled, “System and Method for Determining Dielectrophoresis Crossover Frequencies,” which are incorporated herein by reference in their entirety.

Embodiments of the invention directed to the apparatus and methods that take advantage of the crossover frequency, which is defined as the frequency where the DEP force makes the transition from a negative to a positive force and is dependent on cell or particle and medium conductivity and permittivity. After determining the crossover frequency for a particular target particle, a frequency may then be applied to enrich the fluid specimen for those particular target particles. For example, the applied frequencies of breast, lung, and ovarian cancer cells, solid tumor cancer cells or sarcomas may be less than about 100 kHz, preferably about 1 kHz-about 85 kHz, more preferably about 65 kHz-about 85 kHz compared to about 90 kHz-about 140 kHz for major peripheral blood cell types at an eluate buffer conductivity of about 30 mS/m, for example. Fluid specimens from a subject having a liquid or fluid cancer, including but not limited to leukemias, may be enriched for those cancer cells by applying a frequency of less than about 130 kHz, preferably about 50 kHz to about 130 kHz, more preferably about 75 kHz to about 125 kHz, while fetal or stem cells may be enriched by applying a frequency of less than about 130 kHz, preferably about 80 kHz to about 130 kHz.

The difference between target cancerous and non-target, non-cancerous cells in applied frequencies forms the basis for enriching, for example, circulating T cells (CTCs) from a complex mixture or suspension of cells, and is applicable to a wide variety of cancer types. For example, when an intermediate frequency in the range of about 65 kHz-about 85 kHz is applied, the target cancer cells are under the influence of a positive, attractive DEP force, while the non-target healthy blood cells are repelled into the fluid flow by a negative DEP force, resulting in separation of the target cancer cells from the non-target blood cells.

In an embodiment of the invention, a method for obtaining or isolating target particles of interest that is both high throughput and particle-labelling independent, and preferably automated utilize particles that may be biological or non-biological, including, for example, rare cancer target cells. This approach is based on dielectrophoresis (DEP) in a continuous flow microfluidic chamber for modification- or label-independent (e.g., antibody independent) obtainment of target particles by separating, and collecting the target particles, for example, rare CTCs and cancer stem cells from a sample of blood containing both target cells and non-target cells. By obtaining rare cancer target cells from a sample of whole blood without modification allows subsequent analyses of the target cancer cells for the characterization of the cancer disease state.

One embodiment of the disclosure relates to a method of obtaining or isolating target particles using continuous flow dielectrophoresis, comprising the steps of: (a) introducing an elution buffer continuously through a chamber of a height (H) at a controlled eluate flow rate (Fe) at a first, buffer inlet at one end of the chamber; (b) introducing a sample suspension comprising target particles and non-target particles to a chamber of height (H) at a continuous controlled infusion flow rate (Fi) at a second, sample suspension inlet at one end of the chamber, where the sample suspension reaches an injection height (hi), and where the first inlet and the second inlet are at the same end of the chamber; (c) applying dielectric forces from at least one electrode of the chamber to the target particles and the non-target particles of the sample suspension, where the at least one electrode is placed on a horizontal plane perpendicular to a vertical plane of the height of the chamber, where positive forces attract the target particles to the electrodes and negative forces repel the non-target particles from the electrodes; thereby separately obtaining the target particles and the non-target particles. The height of the chamber may also be the height (H) of the laminar fluid flow path, and more specifically, the flow profile may be a hydrodynamic parabolic flow profile, where at half the height of the laminar fluid flow path (H½), the flow velocity is at a peak flow velocity. Two outlets are located at the opposite end of the chamber. Preferably, the target particles traveling closest to the electrode, where the target particles reach a skim height (hs) and at a controlled withdrawal flow rate (Fw) exit the chamber through a first outlet of the chamber. The remaining non-target particles exit at a controlled excess flow rate (Fx) through a second outlet of the chamber, where the outlets are downstream of the inlets. The obtained target particles may undergo further analyses; whereas, the non-target particles may be discarded or other subpopulations of the non-target particles may be obtained by repeating the method under the appropriate conditions.

A preferred embodiment is directed to a method for the diagnosis, prognosis, screening, and treatment of a subject, wherein the subject is healthy or suffering from a disease or condition comprising the steps of:
a. obtaining a fluid specimen from said subject, wherein said fluid specimen comprises target cells of interest and non-target cells;
b. adding said fluid specimen into a flow chamber;
c. enriching viable target cells of the fluid specimen; and
d. analyzing the viable target cells, thereby enabling diagnosis, prognosis, screening, and treatment of the subject.
   In this embodiment, the enrichment step may more specifically comprise:
applying an electric field from a source to a sample containing the target cells;
identifying a path of movement of the target cell away from an electric field source;
adjusting the electric field so that the target cell approximately pauses movement;
adjusting the electric field so that the target cell changes direction and moves generally toward the electric field source;
identifying a dielectrophoretic cross-over frequency of the target cell; and
applying a target frequency based on the dielectrophoretic cross-over frequency of the target cell sufficient to enrich viable target cells of interest.

### Analyses

Various types of analyses can be performed on the target cells recovered from a fluid sample that enable protein, phenotypic, morphologic, genetic or chromosomal characterization thereof at different levels of resolution and sensitivity and according to the diagnostic purpose of the study, in accordance with what has been described previously.

Another embodiment may be directed to a method of analyzing a fluid specimen from a subject, wherein the subject is healthy or suffering from a disease or condition comprising the steps of:
a. obtaining a fluid specimen from said subject, wherein said fluid specimen comprises target cells of interest and non-target cells;
b. adding said fluid specimen into a flow chamber;
c. enriching viable target cells of said fluid specimen; and
d. analyzing said viable target cells.
   In this embodiment, the enrichment step may more specifically comprise:
applying an electric field from a source to a sample containing the target cells;
identifying a path of movement of the target cell away from an electric field source;
adjusting the electric field so that the target cell approximately pauses movement;
adjusting the electric field so that the target cell changes direction and moves generally toward the electric field source;
identifying a dielectrophoretic cross-over frequency of the target cell; and
applying a target frequency based on the dielectrophoretic cross-over frequency of the target cell sufficient to enrich viable target cells of interest.

A further embodiment may be directed to a method of analyzing a target cell population comprising the steps of:
a. obtaining a fluid specimen from a subject suffering from a disease or condition, wherein said fluid specimen comprises target cells of interest and non-target cells;
b. applying dielectrophoresis field-flow assist technology to said fluid specimen;
c. obtaining a viable target cell population from said dielectrophoresis field-flow-applied fluid specimen; and
d. analyzing said viable target cell population.
   In this method of analysis, the dielectrophoresis field-flow assist technology comprises:
applying an electric field from a source to a sample containing the target cells;
identifying a path of movement of the target cell away from an electric field source;
adjusting the electric field so that the target cell approximately pauses movement;
adjusting the electric field so that the target cell changes direction and moves generally toward the electric field source;
identifying a dielectrophoretic cross-over frequency of the target cell; and
applying a target frequency based on the dielectrophoretic cross-over frequency of the target cell sufficient to obtain viable target cells of interest.

For example, it is possible to proceed to sequencing of CTCs taken from subjects or patients presenting metastases in order to detect the presence of mutations of, for example, the K-RAS gene.

In the case of prostate cancer, it is possible to evaluate the presence of deletions on CTCs by means of single-cell whole-genome amplification.

Again, in the case of cancers of unknown primary origin (CUPs), it is possible to identify the original tissue via the genetic profile and/or the profile of expression of the CTCs, thus obtaining information that is fundamental for the choice of the most appropriate therapy.

In the case of disseminated tumor cells, then, it is possible to perform analysis of gene expression on single CTCs and/or DTCs in order to identify prognostic indicators and potential therapeutic targets.

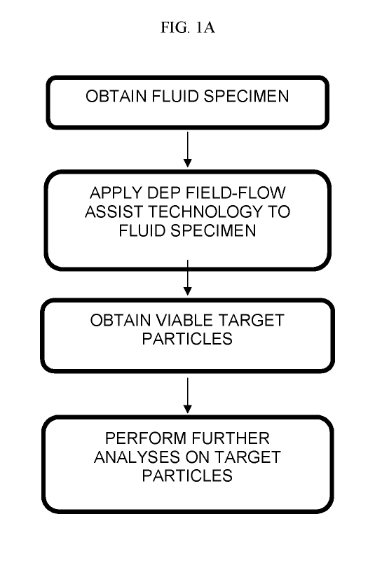

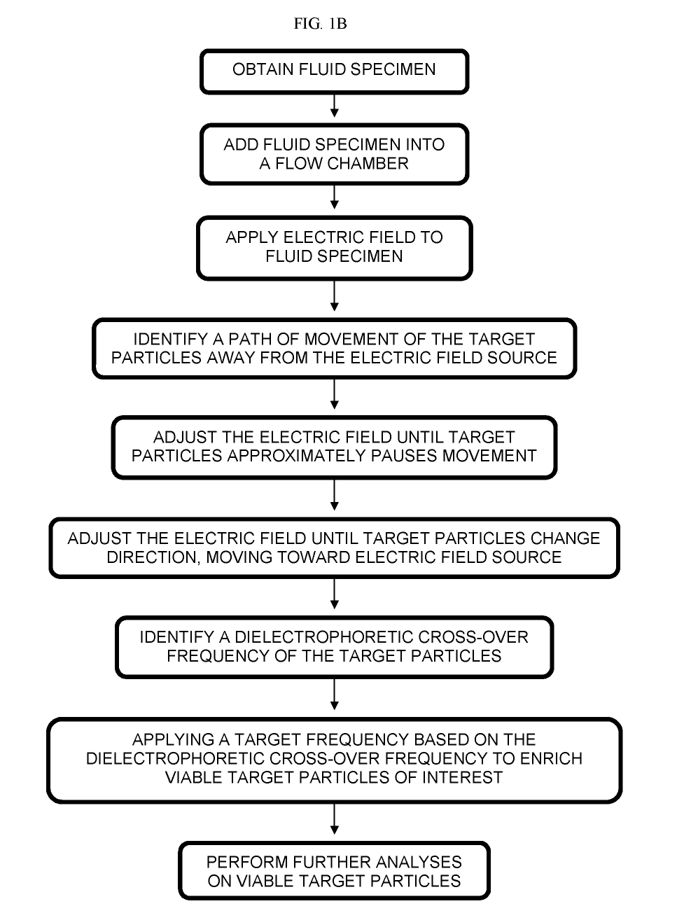

By way of non-limiting examples, described here, are preferential embodiments of the method according to the present invention in the flowcharts represented in FIG. 1.

### Applications

Obtaining a specimen of target cells in a viable, unaltered state in sufficient quantities, renders feasible diagnostic pathways for a plurality of conditions that, otherwise, could not be analyzed accurately, reliably, and precisely. Molecular analyses on the recovered cells may be performed using a variety of techniques, including, by way of non-limiting examples: gene expression analyses: RT-PCR, single-cell gene expression, digital PCR; methylation analyses: quantitative methylation analysis (e.g., pyrosequencing), methylation-sensitive single-nucleotide primer extension (MS-SNuPE), Combined Bisulphite Restriction Analysis of DNA (COBRA), bisulphite-genomic-sequencing PCR (BSP), methylation-specific PCR (MSP); real-time PCR; end-point PCR; analyses of microsatellites by, for example, Quantitative Fluorescent PCR (QF-PCR); comparative genomic hybridization (CGH); array CGH; sequencing, for example, for the identification of mutations; and Next-generation sequencing.

Methods may include providing rare target cells and their use in rare cell based companion diagnostics, cancer diagnoses, cancer metastasis or other disease state, rare cell-based prognosis of cancer or other disease state, and assays or test kits to predict drug response or to direct therapy. The methods and systems of the invention enable generation of patient-specific reagents such as chemical compounds, drugs, vaccines, oligonucleotides or other molecules using rare cells.

Other embodiments may be directed to methods for treating a subject suffering from disease, such as for example a cancer or degenerative disease. Viable target particles of interest may be enriched as a homogeneous population from a flow chamber using specific applied frequencies. Biological material may be extracted from these viable cells, for example, protein, DNA, RNA, and the like, and used for treatment, diagnostic, prognostic clinical testing, and screening. For example, FISH technology may be applied to the DNA of the enriched cell population to determine chromatin alterations. Genetic material, for example, RNA or DNA, may be transfected into and used to stimulate host immune cells in culture. When the modified immune cells are returned to the host, an immune response is elicited such that the host identifies the immune cells expressing the cancerous genetic material as foreign, and thereby provides a more effective treatment for cancer patients where the clinical outcome is more favorable for the patients. The viable target cells may be taken in culture for expansion to evaluate drug sensitivity or resistance. Further, the target cells may be used to generate a PDx tumor animal model which can also be used to tailor patient-specific therapy by screening various drugs in the animal model prior to administration to the patient.

The genetic material from viable target cells and subsets of cell populations obtained at specific applied frequencies enables the enrichment of, for example, metastatic cancer cells that are resistant and causing tumor invasion (metastases). The genetic material from these cells is more effective in treating cancer patients and the clinical outcome is more favorable to the patients.

Additional methods include separation of fetal cells, including fetal stem cells, and methods of using different categories and utilities for using the fetal cells separated by the methods of the invention.

Embodiments of the invention may be directed to a method for separating a target particle in a fluidic sample using dielectrophoresis in a flow chamber, where fluidic samples should be consistent of blood, any bodily fluids including urine, ascites fluid, mucus, or target particles including exosomes. Fluidic samples may also include cells modified by external chemical additions, e.g., cancer cells are exposed to drugs when patients are treated. These drugs alter the chemical state of the cell and the dielectrophoresis or flow chamber claims enables capture of these cells compared to other technologies because the DEP-field-flow technology described here uses electric charge capacitance distributed across the cell to attract the cells to a collection port at the bottom of the chamber. One advantage of using this method is that the technique does rely on antigen or protein expression on the cell surface such as immunomagnetic antibody capture technologies, which is particularly advantageous since these antigens are lost during drug treatment.

The biophysical characteristic changes that occur to cells exposed to cancer therapeutics include protein downregulation, cell resistance and proliferation of resistance cells in circulation. The cell properties can change and cause resistance and mutations to be upregulated. The flow chamber technology can capture cells compared to other technologies even though the cells have been chemically altered.

The flow chamber enables isolation of cancer cells that are responsible for metastatic cancer invasion. These cells can be isolated over a wide range of frequencies from about 10 kHz to about 500 kHz, preferable about 30 kHz to about 100 kHz. For example, a frequency of about 45 kHz to about 65 kHz can be used to isolate cancer stem cells or metastatic cells that cause tumor assaults and are suspended in a fluid buffer medium of electrical conductivity of about 30 mS/m (Gupta et al, Biomicrofluidics, Vol 6, 024133, 2012). The DEP-Field Flow assist technology used here enables capture of these specific subsets of cells which carry increased chromosomal alterations as detected by FISH or sequencing. These subsets of cells enable a diagnostic test because the enriched fraction of cells is the relevant cell type for diagnostic evaluation and correlates with clinical outcome, i.e., prognosis, overall survival, progression free survival.

Other embodiments may be directed to a microfluidic chamber which enables the separation or isolation of cells based on viability in contrast to other separation technologies which isolate both dead and viable cells and other foreign bodies yielding a less pure sample. Pure subsets of cancer stem cells can be isolated by the flow chamber and used for downstream diagnostic purposes. Another disadvantage of other technologies is that they do not allow for diagnostic or therapeutic applications because the impure sample or mixture of cells obtained include dead cells and dead cells are not relevant for medical testing or clinical use.

Whereas, in embodiments of the invention, the methods of isolating target particles isolates viable cells (dependent on cell membrane to be intact) or target particles creating a sample of cells or target particles that is about 100% pure. The pure sample enables subsequent discovery and molecular characterization for therapeutic or diagnostic and prognostic use of the cellular material. Cellular material that can be used from the disclosed isolated cells includes DNA, RNA, protein, or other foreign bodies (chemicals, vehicles for penetration, metabolism, etc.) that can be attached to the target cell or particle of interest.

Protein material from subsets of cells from the flow chamber enables drug target discovery of rare cancer cells. Because the cells are viable and pure this allows for detection of antigens for developing therapeutics that are more effective against killing a specific type of cell that leads to cancer invasion and decreased survival of the patient, decreased progression free survival.

Genetic nucleic, including DNA and RNA, material from subsets of cells isolated using the microfluidic chamber and DEP field-flow assist technology allow specific cell types to detect or extract DNA and RNA for various therapeutic, diagnostic, and prognostic applications. The viable cells are key for improving therapeutic efficacy to improve patient survival. Extracting DNA or RNA for subsequent manipulation by other processes including cell therapy, genetic analysis including fluorescence in situ hybridization (FISH), targeted sequencing for mutations or determination of gene expression of target oncogenes or next gene sequencing based analysis to determine oncogene drivers of metastatic disease. The information obtained from such cell types can guide appropriate therapy by selection of drugs or enable the genetic material to be used to teach immune cells of a host and improve over survival. Additionally, once isolated, these target cells in a homogeneous population may be used for reprogramming stem cells in culture and return them to the patient. reprogramming or the material may be used to stimulate a certain type of cell for reprogramming, for example, regenerating myelin sheath. The DEP field-flow assist technology is more effective than other technologies because the relevant cell populations cannot be captured in a real-time biological homogenous and viable state.

For previously or currently used technologies utilizing magnetic separation such as for example, CellSearch® demonstrating that there is a correlation of survival have not shown that the captured cells are cancer related. The DEP field-flow assist flow chamber isolates known cancer cells relevant for therapeutic and diagnostic testing. This is demonstrated across all cancer types by showing detection of chromosomal alterations at various chromosomes specific for each cancer type, including but not limited to breast, ovarian, prostate, lung, and the like, or by detection of mutations through PCR or Next gen sequencing applications. Additionally the inventive method for isolating cells are proven to be relevant through the ability to take the captured cells from the flow chamber and grow human tumors in immune compromised mouse models.

The flow chamber including DEP field-flow assist technology enables enrichment of a large volume of target cells, thereby allowing separation of specific cell types by applying particular frequencies, for example, about 10 kHz to about 500 kHz frequency, some target cells may be isolated by applying a frequency of about 40 kHz to about 70 kHz frequency, flow rate of about 10 microliters/min to about 40 microliters/min, and osmotic concentration (osmolarity) of about 280 mOsm/L to about 320 mOsm/L. It is the combination of these parameters that enable effective separation of the relevant cancer stem cells or target cells depending on the disease or application e.g, fetal cell isolation.

The target particles isolated by the DEP field-flow assist method described here are different based on their physical morphology, genetics, and chemically. For example, the cells are viable and structurally isolated by various biochemical features, including proteins, DNA mutations. Viable cells carry the genetic and mutation information that can be subsequently isolated and analyzed for diagnostic therapeutic prognostic purposes to drive treatment decisions based on oncogene specific targets and clinically improve patient outcome. The cells may carry genetic mutations, selected by specific frequencies in the chamber and using the operating parameters as noted above. These cells are critical for mapping the oncogenes that are driving tumor disease progression. In fetal cells, this information from these specific cell types can be used to evaluate potential targets for birth defects. Moreover, cells are chemically altered by exposure to environmental factors, such as for example, chemicals, toxicants or cancer therapeutics or in fetal cells, drug exposure may cause genetic mutations. The DEP field-flow assist technology used here can effectively be used to isolate these specific cell subsets, separated from non-relevant cells thereby enabling downstream therapeutic or diagnostic uses. These cells may have lost the ability to be captured efficiently by other systems, e.g., loss of external proteins or changes to the cell density do not enable these cells to purely isolated by other technologies.

Embodiments of the invention are directed to the use of the FISH technique to obtain purified cancer cells by identifying various chromosomal genetic alterations in the various cancer types. The chromosome aberrations are specific for different disease types, for example, lung cancer detecting EML4ALK. A high concordance in detecting EGFR activating mutations in NSCLC CTCs may be compared to tumor biopsies. The cells of interest are also pure enough to generate tumors in mouse models when transplanted into the mouse models, although some supporting stromal cells and cancer stem cells may stimulate this process as well.

### EXAMPLES

The following Examples of the invention are provided only to further illustrate the invention, and are not intended to limit its scope.

### Example 1

### Fetal Cell Analysis

Oftentimes prenatal testing is performed to obtain important information about a baby's health before birth. Generally, amniocentesis is performed which requires the extraction of a small amount of amniotic fluid from the sac surrounding the fetus by fine needle insertion. Prenatal testing may be performed to check for certain types of birth defects, including a chromosomal abnormality such as for example Down syndrome. However, amniocentesis is an invasive technique and presents a risk for both the mother and her baby. The mother may suffer from a miscarriage, an infection, or preterm labor, and an injury to the baby may occur.

In order to determine whether or not a fetus has a genetic disorder or defect, a fluid specimen such as a blood sample, may be obtained from the mother because maternal blood contains both maternal and fetal cells. Instead of performing an invasive procedure such as an amniocentesis, a maternal blood specimen may be collected since it contains both the target fetal cells and the non-target maternal cells. About 30 milliliters of blood is collected from the mother. The maternal blood sample may be subjected to DEP field-flow assist technology by inserting the specimen into the flow chamber for fetal cell separation and isolation from the maternal cells. The fetal cell separation protocol employs a voltage signal frequency of about 70 kHz frequency, flow rate of about 10 microliters/min to about 40 microliters/min, and an osmotic concentration (osmolarity) of about 280 mOsm/L to about 320 mOsm/L. Upon obtaining a population or aggregate of isolated fetal cells, the fetal cells may undergo genetic testing for Down syndrome, sickle cell disease, cystic fibrosis, muscular dystrophy, Tay-Sachs disease, neural tube defects, and the like. Embryonic blood from cord may be collected and frozen for future use should the patient need the blood for any ailments or diseases diagnosed at a later time. For example, the embryonic blood fluid specimen may be enriched for target stem cells for future diagnostics. Another application of these enriched target stem cells may be to reprogram them in culture for proliferation and differentiation into specific cells that may be useful in cell therapy or regenerative medicine.

### Example 2

### Breast Cancer Circulating Tumor Cell Analysis

DEP field-flow assist technology may similarly be used to diagnose a subject having a family history of breast cancer. A blood specimen (about 4 milliliters to about 8 milliliters) may be obtained from an undiagnosed subject and further undergo DEP or hybrid DEP techniques to separate the breast cancer CTCs from the other components of the blood specimen. Since normal, healthy subjects do not have CTCs, an assessment of whether the subject has any CTCs provide early diagnosis for breast cancer.

By using a frequency of about 65 kHz, breast cancer cells are positively attracted towards the electrode surface while normal blood cells are negatively repelled away from the electrode surface. Using this DEP-FF assist phenomena, the breast cancer cells can effectively be separated from normal cells for analysis. After enrichment using DEP-FF assist technology, the cancer cells may be analysed phenotypically or genetically to identify the type, stage, or mutation status of the breast cancer.

The content of all patents, patent applications, published articles, abstracts, books, reference manuals and abstracts, as cited herein are hereby incorporated by reference in their entireties to more fully describe the state of the art to which the disclosure pertains.

All of the features disclosed in this specification may be combined in any combination, except combinations where at least some of such features and/or steps are mutually exclusive. In particular, the preferred features of the invention are applicable to all aspects of the invention and may be used in any combination. Likewise, features described in non-essential combinations may be used separately (not in combination).

It will be appreciated that many of the features described above, particularly of the preferred embodiments, are inventive in their own right and not just as part of an embodiment of the present invention. Independent protection may be sought for these features in addition to or alternative to any invention presently claimed.

It should be understood that the foregoing description is only illustrative of the present disclosure. Various alternatives and modifications can be devised by those skilled in the art without departing from the disclosure. Accordingly, the present disclosure is intended to embrace all such alternatives, modifications and variations that fall within the scope of the appended claims.

1. A method for the diagnosis, prognosis, screening, and treatment of a subject, wherein the subject is healthy or suffering from a disease or condition comprising the steps of:
a. obtaining a fluid specimen from said subject, wherein said fluid specimen comprises target cells of interest and non-target cells;
b. adding said fluid specimen into a flow chamber;
c. enriching viable target cells of the fluid specimen; and
d. analyzing the viable target cells, thereby enabling diagnosis, prognosis, screening, and treatment of the subject.

2. The method according to claim 1, wherein the enriching step comprises:
applying an electric field from a source to a sample containing the target cells;
identifying a path of movement of the target cell away from an electric field source;
adjusting the electric field so that the target cell approximately pauses movement;
adjusting the electric field so that the target cell changes direction and moves generally toward the electric field source;
identifying a dielectrophoretic cross-over frequency of the target cell; and
applying a target frequency based on the dielectrophoretic cross-over frequency of the target cell sufficient to enrich viable target cells of interest.

3. The method according to claim 2, wherein the step of enriching viable target cells results in a diagnostic specimen having sufficient sensitivity to detect at least one target cell.

4. The method according to claim 3, wherein the enriching step occurs using dielectrophoretic field-flow assist technology.

5. The method according to claim 4, wherein said flow chamber is a microfluidic chamber.

6. The method according to claim 5, wherein said target cells are enriched on the basis of cell parameters evaluated in a fluidic state.

7. The method according to claim 6, wherein said target cells are enriched based on cell parameters selected from the group consisting of:
a. mass density;
b. morphology;
c. electrical properties;
d. chemical properties;
e. mechanical properties;
f. expression of surface antigens;
g. expression of intracytosolic antigens;
h. dielectric properties;
i. charge capacitance;
l. optical properties;
m. geometrical properties;
n. magnetic properties; and
o. combinations thereof.

8. The method according to claim 7, wherein said target cells are enriched on the basis of dielectric properties.

9. The method according to claim 8, wherein said target cells for a specific disease have a unique charge capacitance, are metastatic in nature, or potentially resistant to therapies if detected.

10. The method according to claim 9, wherein the target cells are enriched without a label.

11. The method according to claim 10, wherein the target frequency comprises a range of target frequencies sufficient to enrich the target cells of interest.

12. The method according to claim 11, wherein the range of target frequencies for sarcomas is about 1 to about 85 kHz.

13. The method according to claim 11, wherein the range of target frequencies for a fluid cancer is about 50 kHz to about 130 kHz.

14. The method according to claim 11, wherein the range of target frequencies for stem cells or fetal cells is about 80 kHz to about 130 kHz.

15. The method according to claim 11, wherein the analyzing step comprises diagnostic cells labeled with genetic probes or protein clinical markers for prescribing a medical treatment.

16. The method according to claim 15, wherein the probes or markers are directed to fluorescence in situ hybridization (FISH) probes tag.

17. The method according to claim 15, wherein labeling may be used with at least one antibody for a specific marker to identify specific subsets of cells required for diagnostic monitoring.

18. The method according to claim 17, wherein said at least one antibody recognizes an antigen selected from the group consisting of epithelial, epithelial-mesenchymal, mesenchymal, and stem cells.

19. The method according to claim 18, wherein said step of enriching viable target cells uses a downstream apparatus for further characterization of protein, DNA, or RNA content of the viable target cells.

20. The method according to claim 19, wherein said step of enriching occurs based on dielectric properties of target cancer cells compared to normal, non-target cells.

21. The method according to claim 20, wherein said viable target cells are cells selected from the group consisting of circulating tumor cells, disseminated tumor cells, stem cells, cancer stem cells, epithelial cells, mesenchymal cells, epithelial-mesenchymal transition cells, fetal cells, and abnormal supporting cells for tumor assault.

22. The method according to claim 21, wherein the enriching step separates the target cells that are nucleated from the non-target cells that are non-nucleated and enriches a population of the nucleated target cells.

23. The method according to claim 18, wherein the enriching step occurs by positively, negatively, or positively and negatively selecting target cells of interest using magnetophoresis or pre-labeled target particles.

24. The method according to claim 23, wherein said enriching step comprises at least two enrichment steps performed within the same said flow chamber used for performing the enrichment step.

25. The method according to claim 24, wherein said flow chamber comprises a plurality of different chambers, separated from one another, and hydraulically connected, delimited on at least one face by a single chip or by a plurality of separate chips.

26. The method according to claim 25, wherein said step of analyzing comprises a technique selected from the group consisting of:
a. sequencing;
b. microsatellite analysis;
c. comparative genomic hybridization (CGH);
d. array CGH;
e. end-point PCR;
f. real-time PCR;
g. methylation analysis selected from the group consisting of: quantitative methylation analysis with pyrosequencing, bisulphite-genomic-sequencing PCR (BSP), methylation-specific PCR (MSP);
h. digital PCR;
i. Next-gen sequencing; and
j. combinations thereof.

27. The method according to claim 26, wherein said protein, DNA, RNA, or combinations thereof of viable target cells are analyzed.

28. The method according to claim 27, wherein the target cells comprise fetal cells.

29. The method according to claim 28, wherein enriching target cells comprises aggregating cancer cells of at least one population of cells.
cells comprises at least one type of viable circulating cancer cells or disseminated cancer cells.

31. The method according to claim 29, wherein said fluid specimen is added to the flow chamber at a flow rate of about 20 microliters/minute to about 40 microliters/minute.

32. The method according to claim 31, wherein the target cells are isolated at a frequency ranging from about 10 kHz to about 500 kHz.

33. The method according to claim 32, wherein the isolated target cells have a purity ranging from about 90% to about 100%.

34. The method according to claim 33, wherein said purity is about 100%.

35. A method of analyzing a fluid specimen from a subject, wherein the subject is healthy or suffering from a disease or condition comprising the steps of:
a. obtaining a fluid specimen from said subject, wherein said fluid specimen comprises target cells of interest and non-target cells;
b. adding said fluid specimen into a flow chamber;
c. enriching viable target cells of said fluid specimen; and
d. analyzing said viable target cells.

36. The method according to claim 35, wherein the enriching step comprises:
applying an electric field from a source to a sample containing the target cells;
identifying a path of movement of the target cell away from an electric field source;
adjusting the electric field so that the target cell approximately pauses movement;
adjusting the electric field so that the target cell changes direction and moves generally toward the electric field source;
identifying a dielectrophoretic cross-over frequency of the target cell; and
applying a target frequency based on the dielectrophoretic cross-over frequency of the target cell sufficient to enrich viable target cells of interest.

37. The method according to claim 36, wherein the analyzing step comprises performing a technique selected from the group consisting of:
a. drug target discovery of rare cancer cells;
b. detection of antigen for developing therapeutics that are more effective against killing a specific type of cell that leads to cancer assault and decreased survival of said subject or decreased progression free survival;
c. detecting or extracting DNA, RNA, or DNA and RNA using specific cell types for therapeutic, diagnostic, or prognostic applications;
d. cell therapy;
e. genetic analyses;
f. targeted sequencing for mutations;
g. determining gene expression of target oncogenes;
h. next-generation sequencing-based analysis to determine oncogene drivers of metastatic disease;
i. diagnosing cancer, cancer metastases, or cancer stage;
j. prognosing cancer, cancer metastases;
k. predicting drug response for directing therapy;
l. early detection of cancer or disease;
m. for use in regenerative medicine and
i. combinations thereof.

38. The method according to claim 35, wherein the enriching step occurs using dielectrophoretic field-flow assist technology.

39. A method of analyzing a target cell population comprising the steps of:
a. obtaining a fluid specimen from a subject suffering from a disease or condition, wherein said fluid specimen comprises target cells of interest and non-target cells;
b. applying dielectrophoresis field-flow assist technology to said fluid specimen;
c. obtaining a viable target cell population from said dielectrophoresis field-flow-applied fluid specimen;
d. analyzing said viable target cell population.

40. The method according to claim 39, wherein the dielectrophoresis field-flow assist technology comprises:
applying an electric field from a source to a sample containing the target cells;
identifying a path of movement of the target cell away from an electric field source;
adjusting the electric field so that the target cell approximately pauses movement;
adjusting the electric field so that the target cell changes direction and moves generally toward the electric field source;
identifying a dielectrophoretic cross-over frequency of the target cell; and
applying a target frequency based on the dielectrophoretic cross-over frequency of the target cell sufficient to obtain viable target cells of interest.

41. The method according to claim 40, further comprising enriching said obtained target cell population.
